(19) United States
(12) Patent Application Publication
(10) Pub. No.: US 2026/0355133 A1
(43) Pub. Date: Nov. 12, 2026

(54) COMPOSITIONS AND METHODS FOR TREATING CANNABINOID HYPEREMESIS SYNDROME

(71) Applicant: Joseph Habboushe, New York, NY (US)

(72) Inventor: Joseph Habboushe, New York, NY (US)

(21) Appl. No.: 19/357,803

(22) Filed: Oct. 14, 2025

Related U.S. Application Data
(63) Continuation of application No. 18/643,431, filed on Apr. 23, 2024, now abandoned.
Application No. 18/643,431 is a continuation of application No. 17/710,641, filed on Mar. 31, 2022, now abandoned.
Application No. 17/710,641 is a continuation of application No. 16/720,511, filed on Dec. 19, 2019, now abandoned.
(60) Provisional application No. 62/782,616, filed on Dec. 20, 2018.

(51) Int. Cl. A61K 31/165; A61K 31/047

(52) U.S. Cl. CPC A61K 31/165; A61K 31/047

(57) ABSTRACT

Provided are compositions and methods for treating cannabinoid hyperemesis syndrome (CHS). One embodiment of the disclosed method entails administering a marijuana-derived cannabinoid composition in combination with at least one non-cannabinoid TRPV1 modulator. Methods of using TRPV1 agonists or endocannabinoid activators such as nicotine for treating CHS are also provided.

### CROSS REFERENCE TO RELATED APPLICATIONS

This application is a continuation of U.S. application Ser. No. 18/643,431, filed on Apr. 23, 2024, which is a continuation of U.S. application Ser. No. 17/710,641, filed on Mar. 31, 2022, which is a continuation of U.S. application Ser. No. 16/720,511, filed on Dec. 19, 2019, which claims the benefit under 35 U.S.C. § 119 (e) of U.S. Provisional Application Ser. No. 62/782,616 filed Dec. 20, 2018, the content of which is incorporated by reference in its entirety into the present disclosure.

### BACKGROUND

Cannabinoid hyperemesis syndrome (CHS) is a condition of cyclic nausea, vomiting, and abdominal pain in chronic cannabis users without other identifiable etiologies. CHS is also associated with compulsive showering in hot water, age younger than 50 years, morning predominance of symptoms, excessive use of cannabis (one or more times per week over at least one year), and cessation of symptoms with cannabis abstinence. While definitive treatment involves abstinence from cannabinoids, various drug therapies have been studied for symptomatic relief in the acute presentation of a patient suffering from cannabinoid hyperemesis syndrome, often in the setting of a hospital emergency department.

The mechanisms of CHS are not clear. Cannabis contains more than 400 different chemicals, of which about 60 are cannabinoids. The pathophysiology of CHS, therefore, is complicated by the complex action of these chemicals throughout the body, both in the central nervous system and in the gastrointestinal system. Various pathogenic mechanistic theories attempting to explain symptoms have been put forward. One hypothesis relates to dose dependent buildup of cannabinoids and related effects of cannabinoid toxicity (the cannabinoid buildup theory). The other hypothesis is based on the functionality of cannabinoid receptors in the brain and particularly in the hypothalamus which regulates body temperature and the digestive system (the hypothalamic theory). However, the mechanisms by which cannabis causes or controls nausea and the adverse consequences of long-term cannabis toxicity remain unknown.

The prevalence of CHS has been increasing, which likely reflects both an increasing awareness of the condition and a rise in cannabis use over the past decade. Although patients with CHS may report escalating cannabis usage, they are usually unaware that cannabis use is the cause of their symptoms.

### SUMMARY

Provided herein are compositions and methods for the treatment and/or prevention of cannabinoid hyperemesis syndrome (CHS). By utilizing the compositions and methods provided herein, patients are able to continue using cannabis while having the symptoms of cannabinoid hyperemesis syndrome (CHS) alleviated and/or preventing cannabinoid hyperemesis syndrome (CHS) from occurring.

In one embodiment, the present disclosure provides an oral combination composition comprising at least one marijuana-derived cannabinoid and a therapeutically effective amount of at least one non-cannabinoid TRPV1 modulator. In certain embodiments, the non-cannabinoid TRPV1 modulator is a TRPV1 antagonist. In certain embodiments, the oral combination composition further comprises at least one TRPV1 agonist. In certain embodiments, the non-cannabinoid TRPV1 modulator is a TRPV1 agonist. In certain embodiments, the oral combination composition further comprises at least one TRPV1 antagonist.

Also provided herein is a marijuana-derived cannabinoid composition, wherein the composition comprises not more than about 5%, or less than about 4%, or less than about 3%, or less than 2% or less than 1%, of marijuana-derived cannabinoids capable of modulating TRPV1.

Pharmaceutical compositions are also provided, for example, suitable for once daily, twice daily, or three times daily administration. The pharmaceutical compositions can be formulated for administration by one or more method, such as oral and/or inhalation.

Also provided herein is a method of treating and/or preventing cannabinoid hyperemesis syndrome (CHS) in a patient, comprising administering to a patient in need thereof, at least one marijuana-derived cannabinoid in combination with a therapeutically effective amount of at least one non-cannabinoid TRPV1 modulator. In certain embodiments, the non-cannabinoid TRPV1 modulator is a TRPV1 antagonist. In certain embodiments, the method further comprises administering to the patient at least one TRPV1 agonist. In certain embodiments, the non-cannabinoid TRPV1 modulator is a TRPV1 agonist. In certain embodiments, the method further comprises administering to the patient at least one TRPV1 antagonist. Methods of using TRPV1 agonists or endocannabinoid activators such as nicotine for treating/preventing CHS are also provided.

The dual administration allows patients to continue to use cannabis, while alleviating, or preventing the onset of, one or more symptoms of CHS. Such administration is especially useful in patients using cannabis products for medicinal purposes, such as to treat or prevent any one or more of the following: nausea and vomiting, wasting syndrome (AIDS), lack of appetite (exhibited in cancer and AIDS patients as well as patients suffering from anorexia nervosa), multiple sclerosis, spinal cord trauma, epilepsy, pain, arthritis (or other musculoskeletal disorders), movement disorders, glaucoma, asthma, hypertension, psychiatric disorders, Alzheimer's, dementia, general inflammation, and/or gastrointestinal disorders.

Another embodiment provides a composition comprising at least one marijuana-derived cannabinoid and a therapeutically effective amount of at least one non-cannabinoid TRPV1 antagonist. In some embodiments, the composition further comprises at least one non-cannabinoid TRPV1 agonist.

Also provided, in one embodiment, is a composition comprising at least one marijuana-derived cannabinoid and a therapeutically effective amount of at least one non-cannabinoid TRPV1 agonist. In some embodiment, the composition further comprises at least one non-cannabinoid TRPV1 antagonist.

Another embodiment provides a marijuana-derived cannabinoid composition which comprises cannabidiol (CBD) and less than about 3%, or less than 2% or less than 1% of a combination of cannabigerol, cannabichromene or cannabidiol acid.

In one embodiment, provided is a method of treating or preventing cannabinoid hyperemesis syndrome (CHS) in a patient, comprising administering to a patient in need thereof, at least one marijuana-derived cannabinoid in combination with a therapeutically effective amount of at least one TRPV1 antagonist. In some embodiments, the TRPV1 antagonist is a competitive antagonist. In some embodiments, the TRPV1 antagonist is selected from capsazepine, JYL-1421, A-425619, BCTC, JNJ-17203212, SB-705498, SB-366791, AMG-9810, MK-2295, and AMG-2674. In some embodiments, the TRPV1 antagonist is a non-competitive antagonist.

In some embodiments, the TRPV1 antagonist is selected from an arginine-rich hexapeptide, methoctramine, AG-489, AG-505, DD-161515, and DD-191515. In some embodiments, the TRPV1 antagonist and marijuana-derived cannabinoid composition are administered concurrently. In some embodiments, the TRPV1 antagonist is administered orally.

In some embodiments, the marijuana-derived cannabinoid is administered via inhalation. In some embodiments, the marijuana-derived cannabinoid is administered orally. In some embodiments, the TRPV1 antagonist and marijuana-derived cannabinoid are orally administered in a combination formulation.

In some embodiments, the method further comprises at least partially activating TRPV1, which can be using nociceptive heat or a TRPV1 antagonist. In some embodiments, the TRPV1 antagonist comprises resiniferatoxin (RTX), menthol or an endovanilloid.

Also provided is a method of treating or preventing cannabinoid hyperemesis syndrome (CHS) in a patient, comprising administering to a patient in need thereof, at least one marijuana-derived cannabinoid and a therapeutically effective amount of at least one TRPV1 agonist. In some embodiments, the marijuana-derived cannabinoid and the TRPV1 agonist are administered concurrently. In some embodiments, the marijuana-derived cannabinoid and the TRPV1 agonist are administered in a fixed dose combination formulation. In some embodiments, the method further comprises orally administering a therapeutically effective amount of at least one TRPV1 antagonist.

In some embodiments, the marijuana-derived cannabinoid composition comprises cannabidiol (CBD) and less than about 3%, or less than 2% or less than 1% of a combination of cannabigerol, cannabichromene or cannabidiol acid. In some embodiments, the method further comprises administering a therapeutic agent for the treatment or prevention of acute cannabinoid hyperemesis syndrome (CHS), such as diethylstilboestrol, oleoylethanolamid, capsazepine, beta agonists, gabapentin and/or arachidonylethanolamine.

Also provided is a method of treating or preventing cannabinoid hyperemesis syndrome (CHS) in a patient, comprising administering to a patient in need thereof a therapeutically effective amount of a TRPV1 agonist. In some embodiments, the TRPV1 agonist is a non-competitive TRPV1 agonist. In some embodiments, the TRPV1 agonist does not compete with cannabidiol in binding to TRPV1. In some embodiments, the agonist is in a long acting and slow release form. In some embodiments, the long acting and slow release form releases no more than half of the TRPV1 agonist within a day. In some embodiments, the TRPV1 agonist is selected from the group consisting of capsaicin, resiniferatoxin, NGX-4010 and ALGRX 4975.

In another embodiment, provided is a method of treating or preventing cannabinoid hyperemesis syndrome (CHS) in a patient, comprising administering to a patient in need thereof a therapeutically effective amount of a TRPV1 upregulator, such as capsazepine, norepinephrine, beta adrenergic agonists, diethylstiboestrol, oleoylethanolamide, and anandamide (AEA).

Also provided is a marijuana-derived cannabinoid composition which comprises at least two compounds selected from the group consisting of tetrahydrocannabinol (THC), tetrahydrocannabinolic acid (THCA), cannabichromene (CBC) and cannabinol (CBN), wherein the total weight percentage of THC, THCA, CBC and CBN in the composition is at least 80%. In some embodiments, the marijuana-derived cannabinoid composition comprises no more than 1% w/w of cannabidiol (CBD), cannabigerol (CBG), cannabigerovarin (CBGV), and tetrahydrocannabivarin (THCV) in combination. In some embodiments, the marijuana-derived cannabinoid composition comprises no more than 0.5% w/w of any of CBD, CBG, CBGV or THCV.

Also provided is a method of treating or preventing cannabinoid hyperemesis syndrome (CHS) in a patient, comprising administering to a patient in need thereof a therapeutically effective amount of an agent that activates the endocannabinoid system (ECS) in the patient. In some embodiments, the agent comprises nicotine. In some embodiments, the nicotine is administered as a patch, a gum, a cream, or an injectable solution.

In some embodiments, the agent comprises capsaicin or resiniferatoxin (RTX). In some embodiments, the capsaicin or RTX is administered as a cream, a capsule, an intranasal formulation, or an injectable solution. In some embodiments, the agent comprises an endocannabinoid.

In some embodiments, the agent is selected from the group consisting of anandamide (AEA), 2-arachidonoylglycerol (2-AG), and a mimetic or activator thereof. In some embodiments, the agent is selected from the group consisting of R(+)-Methanandamide, dihydroxyphenylglycine (DHPG), N-palmitoylethanolamide (PEA), and N-oleoylethanolamide (OEA).

### DETAILED DESCRIPTION

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

The term “about” refers to a variation of ±1%, ±3%, ±5%, or ±10% of the value specified. For example, “about 50” can in some embodiments includes a range of from 45 to 55. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. Also, the singular forms “a” and “the” include plural references unless the context clearly dictates otherwise. Thus, e.g., reference to “a cannabinoid” includes a plurality of such compounds.

As used herein, “treatment” or “treating” is an approach for obtaining a beneficial or desired result, such as a clinical result. For purposes of this disclosure, beneficial or desired clinical results include, but are not limited to, alleviation of a symptom and/or diminishment of the extent of a symptom and/or preventing a worsening of a symptom associated with a disease or condition. For use herein, the beneficial or desired clinical results include, but are not limited to, alleviation of a symptom and/or diminishment of the extent of a symptom and/or preventing a worsening of a symptom associated with marijuana use, or specifically, cannabinoid hyperemesis syndrome (CHS). Preferably, treatment of cannabinoid hyperemesis syndrome a composition or treatment method described herein is accompanied by no or fewer side effects than are associated with currently available therapies for the disease or condition and/or improves the quality of life of the individual. Prevention measures can be taken to individuals or patients who are at risk of developing the disease or symptoms.

As used herein, by “combination therapy” is meant a therapy that includes two or more different compounds or compositions. In some variations, the combination therapy optionally includes one or more pharmaceutically acceptable carriers or excipients, non-pharmaceutically active compounds, and/or inert substances.

As used herein, the term “effective amount” intends such amount of a compound or composition of the disclosure which in combination with its parameters of efficacy and toxicity, as well as based on the knowledge of the practicing specialist should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved.

As used herein, the term “long acting” intends such that a method or composition is given in such an amount and route of administration that there remains enough of the active ingredients in the patient's system sufficient for at least partial relief for a period of greater than four hours, eight hours, sixteen hours, 1 day, 2 days, 4 days, 1 week, 2 weeks, 3 weeks, or 4 weeks. In some embodiments, the long acting composition has a controlled release profile, e.g., not releasing more than half of the active ingredients within two hours, four hours, eight hours, sixteen hours, 1 day, 2 days, 4 days, 1 week, or 2 weeks.

As used herein, the term “agonist” refers to a compound or composition, the presence of which results in a biological activity of a target receptor that is the same as the biological activity resulting from the presence of a naturally occurring ligand for the target receptor, such as, for example, transient receptor potential vanilloid subtype 1 (TRPV1) receptor.

As used herein, the term “partial agonist” refers to a compound the presence of which results in a biological activity of a protein that is of the same type as that resulting from the presence of a naturally occurring ligand for the target receptor, but of a lower magnitude.

As used herein, the term “antagonist” or “inhibitor” refers to a compound or composition, the presence of which results in a decrease in the magnitude of a biological activity of a target receptor. In certain embodiments, the presence of an antagonist results in complete inhibition of a biological activity of a target receptor, such as, for example, the transient receptor potential vanilloid subtype 1 (TRPV1) receptor.

As used herein, the term “carrier” refers to relatively nontoxic chemical compounds or agents that facilitate the incorporation of a compound into cells or tissues.

As used herein, the term “unit dosage form” refers to physically discrete units, suitable as unit dosages, each unit containing a predetermined quantity of active ingredient calculated to produce the desired therapeutic effect. Unit dosage forms may contain a single or a combination therapy.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

It is contemplated that any species of marijuana (i.e., cannabis) can be used in the methods described herein. The cannabis genus (marijuana, weed) is a wind-pollinated dioecious flowering plant that belongs to the Cannabaceae family. Current research shows that the cannabis genus has seven sub species, including but not limited to Cannabis sativa, Cannabis indica, and Cannabis ruderalis-any/all subspecies and any/all crossbreeds thereof and any/all established strains. The therapeutic use of cannabis stretches back to ancient times; it was cultivated in China around 4000BC and is included in the world's oldest pharmacopoeia written by Pen Ts′ao Ching. There are three common subspecies of cannabis: Cannabis sativa (biannual), Cannabis indica (annual) and Cannabis ruderalis (varies), but there are seven subspecies total.

Current research shows that there are over 3,000 established cannabis strains. Growing conditions and genetics influence the characteristics of developing plants and the subsequent chemical characteristics of finished cannabis products. Mature male plants, known as hemp, have minute cannabinoid contents and are typically used to manufacture goods and as an alternative energy source. Hemp may be medicinally beneficial due to its low THC and high CBD chemotypes, which results in low psychoactivity and increased medical viability. The harvested flowers originating from mature female cannabis plants are characteristically higher in phytocannabinoid content and typically possess significant concentrations of other phytochemicals, such as terpenes, that are also of pharmaceutical interest. Cannabinoids and terpenes are secreted by glandular trichomes that occur most abundantly on the floral calyxes and bracts of the female marijuana plant. Dried cannabis flowers are the most basic form of cannabis. Other, more potent, preparations of cannabis include hashish (typically ranging from 20-65% tetrahydrocannabinol (THC)) and hash oil (typically ranging from 50-90% THC). Cannabis has at least 545 distinct compounds that span 20 chemical classes including cannabinoids, terpenes/terpenoids, amino acids, nitrogenous compounds, simple alcohols, aldehydes, ketones, esters, lactones, and acids, fatty acids, steroids, non-cannabinoid phenols, pigments, flavonoids, vitamins, proteins, enzymes, glycoproteins, and hydrocarbons. Cannabinoids and terpenes, in particular, have shown great potential in terms of medicinal value.

The term “marijuana-derived cannabinoid” refers to a naturally occurring cannabinoid isolated from a cannabis source, or synthesized to be analogous to a naturally occurring cannabinoid isolated from a cannabis source. Synthetic cannabinoids can include cannabinoids structurally related to THC, the nonclassical cannabinoids (cannabimimetics) including the aminoalkylindoles, 1,5-diarylpyrazoles, quinolines, and arylsulfonamides as well as eicosanoids related to endocannabinoids. The classical cannabinoids are concentrated in a viscous resin produced in structures known as glandular trichomes. At least 113 different cannabinoids have been isolated from the Cannabis plant. The main classes of cannabinoids from Cannabis are shown below in Table 1.

TABLE 1
Main classes of naturally occurring cannabinoids

| Type | Skeleton | Cyclization |
|---|---|---|
| Cannabigerol-type CBG | 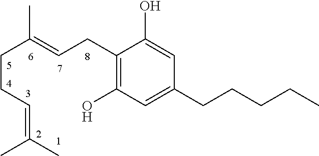 [SMILES: CCCCCc1cc(O)c(C/C=C(\C)CCC=C(C)C)c(O)c1] | 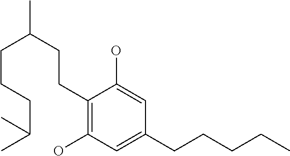 [SMILES: CCCCCc1cc(O)c(CCC(C)CCCC(C)C)c(O)c1] |
| Cannabichromene-type CBC | 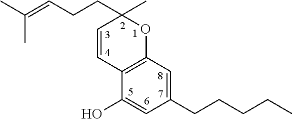 [SMILES: CCCCCc1cc(O)c2c(c1)OC(C)(CCC=C(C)C)C=C2] | 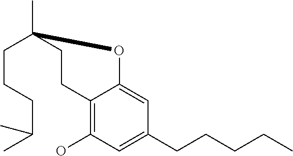 |
| Cannabidiol-type CBD | 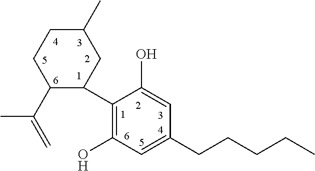 [SMILES: C=C(C)C1CCC(C)CC1c1c(O)cc(CCCCC)cc1O] | 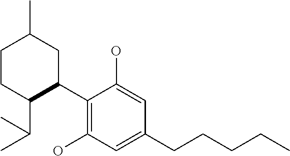 |
| Tetrahydrocannabinol- and Cannabinol-type THC, CBN | 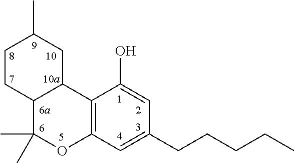 [SMILES: CCCCCc1cc(O)c2c(c1)OC(C)(C)C1CCC(C)CC21] | 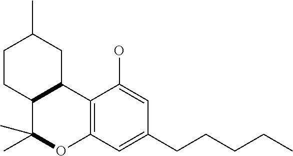 |
| Cannabielsoin-type CBE | 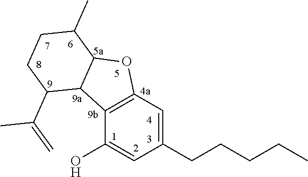 [SMILES: C=C(C)C1CCC(C)C2Oc3cc(CCCCC)cc(O)c3C12] | 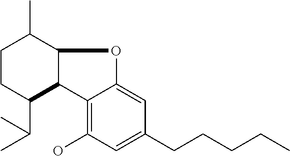 |
| iso- Tetrahydrocannabinol- type iso-THC | 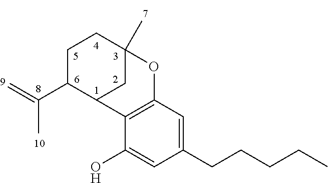 [SMILES: C=C(C)C1CCC2(C)CC1c1c(O)cc(CCCCC)cc1O2] | 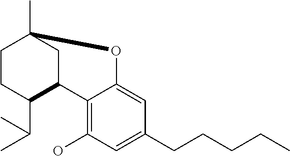 |
| Cannabicyclol-type CBL | 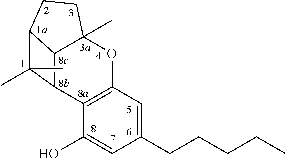 | 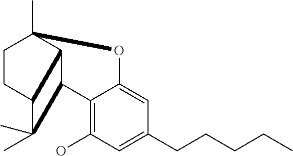 |
| Cannabicitran-type CBT | 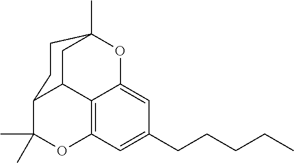 [SMILES: CCCCCc1cc2c3c(c1)OC(C)(C)C1CCC(C)(CC31)O2] | 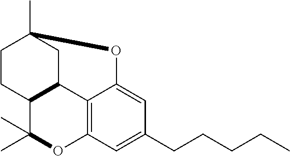 |

Representative marijuana-derived cannabinoids include THC (Tetrahydrocannabinol), THCA (Tetrahydrocannabinolic acid), CBD (Cannabidiol), CBDA (Cannabidiolic Acid), CBN (Cannabinol), CBG (Cannabigerol), CBC (Cannabichromene), CBL (Cannabicyclol), CBV (Cannabivarin), THCV (Tetrahydrocannabivarin), CBDV (Cannabidivarin), CBCV (Cannabichromevarin), CBGV (Cannabigerovarin), CBGM (Cannabigerol Monomethyl Ether), CBE (Cannabielsoin), and CBT (Cannabicitran).

As used herein, the term “TRPV1 modulator” refers to a compound which is capable of modulating transient receptor potential cation channel subfamily V member 1 (TRPV1). As used herein, the term “non-cannabinoid TRPV1 modulator” refers to a compound which is capable of modulating transient receptor potential cation channel subfamily V member 1 (TRPV1) which is not isolated from, or synthetically derived from or based structurally on, a cannabinoid. The TRPV1 modulator can either activate or inhibit TRPV1.

As used herein, the term “TRPV1 antagonist” refers to a cannabinoid compound (ligand or drug) which is capable of binding transient receptor potential cation channel subfamily V member 1 (TRPV1) and blocking or dampening its biological function. As used herein, the term “non-cannabinoid TRPV1 antagonist” refers to a non-naturally occurring cannabinoid compound (ligand or drug) which is capable of binding transient receptor potential cation channel subfamily V member 1 (TRPV1) and blocking or dampening its biological function. The non-cannabinoid TRPV1 antagonist can be a competitive and or a non-competitive TRPV1 antagonist. Antagonists that bind to the agonist binding site, and lock the channel in the closed, nonconductive state are competitive antagonists. In contrast, antagonists that interact with additional binding sites on the receptor structure preventing receptor opening by the agonist or blocking its aqueous pore are non-competitive antagonists.

As used herein, the term “TRPV1 agonist” refers to a compound (ligand or drug) which is capable of binding transient receptor potential cation channel subfamily V member 1 (TRPV1) and producing a biological response. As used herein, the term “non-cannabinoid TRPV1 agonist” refers to a compound (ligand or drug) which is capable of binding transient receptor potential cation channel subfamily V member 1 (TRPV1) and producing a biological response. The non-cannabinoid TRPV1 agonist is not isolated from, or synthetically derived from or based structurally on, a cannabinoid.

### Compositions

Provided herein are compositions and methods for the treatment and/or prevention of cannabinoid hyperemesis syndrome (CHS). By utilizing the compositions and methods provided herein, patients are able to continue using cannabis while having the symptoms of CHS alleviated and/or preventing CHS from occurring.

It is contemplated that when a marijuana product is used, a concomitant use of a TRPV1 antagonist can be useful in reducing the marijuana product's ability to cause CHS. Therefore, in one embodiment, provided is a marijuana product with a TRPV1 antagonist added. The marijuana product can be in any form, such as a smoking product, a vaporizer, a cannabis tea or edibles. In some embodiments, the TRPV1 antagonist is capsazepine. It is contemplated that capsazepine blocks the effect of some TRPV1 agonists (e.g. capsaicin) but not others (e.g., very hot water). Therefore, the use of such a TRPV1 antagonist is particularly advantageous.

In another embodiment, provided is a marijuana product that has reduced amounts of TRPV1 agonists (e.g., CBD, CBG, CBGV and THCV). In some embodiments, the amounts of cannabinoids that do not activate TRPV1 can be enriched. Examples of such cannabinoids include THC, TCHA, CBC and CBN.

In one embodiment, provided is a marijuana product with a TRPV1 agonist added. The marijuana product can be in any form, such as a smoking product, a vaporizer, a cannabis tea or edibles. In some embodiments, the TRPV1 agonist is a non-cannabinoid TRPV1 agonist. In some embodiments, the TRPV1 agonist is a long acting and/or slow release TRPV1 agonist. In some embodiments, the TRPV1 agonist is a non-competitive TRPV1 agonist, such as one that does not compete with cannabidiol in binding to TRPV1.

Before or when CHS occurs, it is contemplated that administration of a TRPV1 agonist can be helpful in ameliorating the symptoms. In some embodiments, a method is provided for treating or ameliorating a CHS symptom by administering to a subject in need a TRPV1 agonist. In some embodiments, the TRPV1 agonist is a non-cannabinoid TRPV1 agonist. In some embodiments, the TRPV1 agonist is a long acting and/or slow release TRPV1 agonist. In some embodiments, the TRPV1 agonist is a non-competitive TRPV1 agonist, such as one that does not compete with cannabidiol in binding to TRPV1.

Prevention or treatment of CHS can also be achieved, it is contemplated, with administration of a TRPV1 upregulator that increases the activity of or sensitize TRPV1. Non-limiting examples of TRPV1 upregulators include capsazepine, norepinephrine, beta adrenergic agonists, diethylstiboestrol, oleoylethanolamide, and anandamide (AEA).

In one embodiment, the present disclosure provides a composition comprising at least one marijuana-derived cannabinoid and a therapeutically effective amount of at least one non-cannabinoid TRPV1 modulator.

In one embodiment, the present disclosure provides a composition comprising at least one marijuana-derived cannabinoid and a therapeutically effective amount of at least one non-cannabinoid TRPV1 antagonist. In some embodiments, the combination is administered orally. In some embodiments, the combination is administered sublingually. In some embodiments, the combination is administered by inhalation. In one embodiment, the non-cannabinoid TRPV1 antagonist is a competitive TRPV1 antagonist. In one embodiment, the non-cannabinoid TRPV1 antagonist is a non-competitive TRPV1 antagonist. In one embodiment, the non-cannabinoid TRPV1 antagonist is not present in isolated from, or synthetically derived from or based structurally on, a cannabinoid.

Exemplary non-cannabinoid TRPV1 antagonists include, but are not limited to, menthol, oleovanillamine, phenylacetylrivanyl, capsazepine, JNJ-17203212, AMG-2674, JYL-1421, BCTC, SB-705498, SB-452533, ABT-102, A-425619, SB-366791, AMG-9810, AMG-0347, SB-782443, GRC 6211, JTS-653, MK-2295, AMG-517, AMG-8163, AMG-8562, SAR-115740, AZD1386, A-452619, A-784168, A-795614, and A-914880.

In certain embodiments, the combination is further administered in combination with at least one non-cannabinoid TRPV1 agonist. Exemplary non-cannabinoid TRPV1 agonists include but are not limited to, capsaicin, resiniferatoxin, NGX-4010 and ALGRX 4975.

In one embodiment, the present disclosure provides an combination composition comprising at least one marijuana-derived cannabinoid and a therapeutically effective amount of at least one non-cannabinoid TRPV1 agonist. Exemplary non-cannabinoid TRPV1 agonists include, but are not limited to, capsaicin and resiniferatoxin. In certain embodiments, the combination is administered in combination with at least one non-cannabinoid TRPV1 antagonist, such as menthol oleovanillamine, phenylacetylrivanyl. capsazepine, JNJ-17203212, AMG-2674, JYL-1421, BCTC, SB-705498, SB-452533, ABT-102, A-425619, SB-366791, AMG-9810, AMG-0347, SB-782443, GRC 6211, JTS-653, MK-2295, AMG-517, AMG-8163, AMG-8562, SAR-115740, AZD1386, A-452619, A-784168, A-795614, or A-914880.

In certain embodiments, provided is a marijuana composition where the naturally occurring components which are capable of modulating TRPV1 have been selectively removed by extraction or isolation. Therefore, in certain embodiments, also provided herein is a marijuana-derived cannabinoid composition, wherein the composition comprises less than about 5%, or less than about 4%, or less than about 3%, or less than 2% or less than 1%, of marijuana-derived cannabinoids capable of modulating TRPV1.

Exemplary cannabinoids capable of modulating TRPV1, include, but are not limited to, cannabichromene (CBC), cannabidiol (CBD), cannabigerol (CBG), cannabinol (CBN), cannabidiol acid (CBDA), cannabigerol acid (CBGA), cannabidivarin (CBDV), cannabigevarin (CBGV), 49-tetrahydrocannabinol (THC), A9-tetrahydrocannabinol acid (THCA), tetrahydrocannabivarin (THCV), and tetrahydrocannabivarin acid (THCVA).

As provided, one embodiment of the disclosure provides a marijuana-derived cannabinoid composition that has reduced amounts of TRPV1 agonists (e.g., CBD, CBG, CBGV and THCV) and/or enriched cannabinoids that do not activate TRPV1 (THC, TCHA, CBC and CBN). In some embodiments, the composition includes at least 50%, 60%, 70%, 75%, 80%, 85%, 90%, 95%, 98% or 99% (w/w) of at least two compounds selected from the group consisting of tetrahydrocannabinol (THC), tetrahydrocannabinolic acid (THCA), cannabichromene (CBC) and cannabinol (CBN). In some embodiments, the composition includes at least three of THC, TCHA, CBC and CBN. In some embodiments, the composition includes all four of THC, TCHA, CBC and CBN.

In some embodiments, the composition includes less (CBD), cannabigerol (CBG), cannabigerovarin (CBGV), and/or tetrahydrocannabivarin (THCV) as compared to the unprocessed marijuana. In one embodiment, the total amount of CBD, CBG, CBGV and THCV in the composition is not greater than about 10%, 5%, 4%, 3%, 2%, 1%, 0.5%, 0.2%, 0.1%, 0.05%, 0.02%, or 0.01% (w/w). In one embodiment, the amount of at least one of CBD, CBG, CBGV or THCV in the composition is not greater than about 10%, 5%, 4%, 3%, 2%, 1%, 0.5%, 0.2%, 0.1%, 0.05%, 0.02%, or 0.01% (w/w).

In certain embodiment, provided is a marijuana-derived cannabinoid composition, wherein the composition comprises less than about 5%, less than about 4%, less than about 3%, less than about 2%, or less than about 1% of cannabidiol. In certain embodiment, provided is a marijuana-derived cannabinoid composition, wherein the composition comprises less than about 5%, less than about 4%, less than about 3%, less than about 2%, or less than about 1% of cannabichromene. In certain embodiment, provided is a marijuana-derived cannabinoid composition, wherein the composition comprises less than about 5%, less than about 4%, less than about 3%, less than about 2%, or less than about 1% of cannabigerol. In certain embodiment, provided is a marijuana-derived cannabinoid composition, wherein the composition comprises less than about 5%, less than about 4%, less than about 3%, less than about 2%, or less than about 1% of cannabinol. In certain embodiment, provided is a marijuana-derived cannabinoid composition, wherein the composition comprises less than about 5%, less than about 4%, less than about 3%, less than about 2%, or less than about 1% of cannabidiol acid. In certain embodiment, provided is a marijuana-derived cannabinoid composition, wherein the composition comprises less than about 5%, less than about 4%, less than about 3%, less than about 2%, or less than about 1% of cannabigerol acid. In certain embodiment, provided is a marijuana-derived cannabinoid composition, wherein the composition comprises less than about 5%, less than about 4%, less than about 3%, less than about 2%, or less than about 1% of cannabidivarin. In certain embodiment, provided is a marijuana-derived cannabinoid composition, wherein the composition comprises less than about 5%, less than about 4%, less than about 3%, less than about 2%, or less than about 1% of cannabigevarin.

In certain embodiment, provided is a marijuana-derived cannabinoid composition, wherein the composition comprises less than about 5%, less than about 4%, less than about 3%, less than about 2%, or less than about 1% of Δ⁹-tetrahydrocannabinol. In certain embodiment, provided is a marijuana-derived cannabinoid composition, wherein the composition comprises less than about 5%, less than about 4%, less than about 3%, less than about 2%, or less than about 1% of Δ⁹-tetrahydrocannabinol acid. In certain embodiment, provided is a marijuana-derived cannabinoid composition, wherein the composition comprises less than about 5%, less than about 4%, less than about 3%, less than about 2%, or less than about 1% of tetrahydrocannabivarin. In certain embodiment, provided is a marijuana-derived cannabinoid composition, wherein the composition comprises less than about 5%, less than about 4%, less than about 3%, less than about 2%, or less than about 1% of tetrahydrocannabivarin acid.

Such compositions can be obtained via standard extraction/isolation of cannabis raw material using any suitable extraction medium, including but not limited to, cold water extraction and dry sift (hash), CO₂, hydrocarbons (including but not limited to: butane, propane, hexane), alcohol (including but not limited to: ethanol, iso-propyl, methanol), and rosin tech (heat extraction)) of cannabis and any/all medically viable compounds found therein (including but not limited to cannabinoids, nitrogenous compounds, amino acids, proteins, enzymes, glycoproteins, hydrocarbons, simple alcohols, aldehydes, ketones and acids, fatty acids, simple esters and lactones, steroids, terpenes, non-cannabinoid phenols, flavonoids, vitamins, and pigments) and its species (including but not limited to Cannabis sativa, Cannabis indica, Cannabis ruderalis—and any/all subspecies and any/all cross breeds thereof and any/all established strains—in both male and female varieties—using both live and dried cannabis plant material) and the subsequent conversion of these extracts into pill-able forms for oral administration (immediate and extended release) is described. In some embodiments, formulations disclosed herein will utilize the use of surfactant(s)/emulsifier(s) and nutritional compounds in order to increase the bioavailability and subsequent absorption of these orally administered cannabis pills/capsules, and the medically viable compounds found therein as a result of a total cannabis extraction, within the mammalian gastrointestinal tract (effectively bypassing the hepatic first pass effect). Varieties will include but will not be limited to: Vegan, kosher, halal, gluten free, extremely potent, CBD rich, low potency, non-decarboxylated (non-psychoactive), live plant material, allergen-free, extended release, very low or sodium free, established cannabis strains, and more.

### Methods of Treatment

Also provided herein is a method of treating and/or preventing cannabinoid hyperemesis syndrome (CHS) in a patient, comprising administering to a patient in need thereof, at least one marijuana-derived cannabinoid in combination with a therapeutically effective amount of at least one non-cannabinoid TRPV1 modulator.

In one embodiment, the non-cannabinoid TRPV1 antagonist is a competitive TRPV1 antagonist. In one embodiment, the non-cannabinoid TRPV1 antagonist is a non-competitive TRPV1 antagonist. In one embodiment, the non-cannabinoid TRPV1 antagonist is not present in isolated from, or synthetically derived from or based structurally on, a cannabinoid. Exemplary non-cannabinoid TRPV1 antagonists for use in the methods described herein include, but are not limited to, menthol, oleovanillamine, phenylacetylrivanyl. capsazepine, JNJ-17203212, AMG-2674, JYL-1421, BCTC, SB-705498, SB-452533, ABT-102, A-425619, SB-366791, AMG-9810, AMG-0347, SB-782443, GRC 6211, JTS-653, MK-2295, AMG-517, AMG-8163, AMG-8562, SAR-115740, AZD1386, A-452619, A-784168, A-795614, and A-914880.

In one embodiment, the TRPV1 antagonist is a competitive antagonist. Exemplary competitive non-cannabinoid TRPV1 antagonists include, but are not limited to, capsazepine, JYL-1421, A-425619, BCTC, JNJ-17203212, SB-705498, SB-366791, AMG-9810, MK-2295, and AMG-2674.

In one embodiment, the TRPV1 antagonist is a non-competitive antagonist. Exemplary non-competitive non-cannabinoid TRPV1 antagonists include, but are not limited to, an arginine-rich hexapeptide, methoctramine, AG-489, AG-505, DD-161515, and DD-191515.

In certain embodiments, the method further comprises administering to the patient at least one TRPV1 agonist. Exemplary non-cannabinoid TRPV1 agonists include but are not limited to, capsaicin, resiniferatoxin, NGX-4010 and ALGRX 4975. The TRPV1 agonist can be administered concurrently with the marijuana-derived cannabinoid and non-cannabinoid TRPV1 antagonist, or just before, or just after, either component.

In certain embodiments, the non-cannabinoid TRPV1 modulator is a TRPV1 agonist. Accordingly, also provided is a method of treating and/or preventing cannabinoid hyperemesis syndrome (CHS) in a patient, comprising administering to a patient in need thereof, at least one marijuana-derived cannabinoid in combination with a therapeutically effective amount of at least one non-cannabinoid TRPV1 agonist. Exemplary non-cannabinoid TRPV1 agonists include but are not limited to, capsaicin, resiniferatoxin, NGX-4010 and ALGRX 4975.

Any of the methods or compositions described herein can be combined with administering a therapeutic agent for the treatment of acute cannabinoid hyperemesis syndrome (CHS), such as diethylstilboestrol, oleoylethanolamid, capsazepine, beta agonists, gabapentin and/or arachidonylethanolamine. In certain embodiments, the method further comprises at least partially activating TRPV1, such as by nociceptive heat (e.g., a hot shower), or with an additional TRPV1 antagonist (e.g., resiniferatoxin (RTX), menthol or an endovanilloid).

Another embodiment of the present disclosure provides methods of treating cannabinoid hyperemesis syndrome (CHS) with a TRPV1 agonist in a long acting and slow release form. The long acting and slow release form, in some embodiments, retains enough of the TRPV1 agonist in the patient's system sufficient for at least partial relief for a period of greater than four hours, eight hours, sixteen hours, 1 day, 2 days, 4 days, 1 week, 2 weeks, 3 weeks, or 4 weeks. In some embodiments, the long acting form does not release more than half of the active ingredients within two hours, four hours, eight hours, sixteen hours, 1 day, 2 days, 4 days, 1 week, or 2 weeks

Treatment of cannabinoid hyperemesis syndrome (CHS) can also be achieved with TRPV1 upregulators that regulate TRPV1 receptor activity or expression, or sensitize TRPV1 receptor. Non-limiting examples include CPZ (capzaizepine) which is a selective TRPV1 antagonist that blocks capsaicin but NOT heat effect or bradykinin effect on TRPV1, norepinephrine, beta-agonists, dES (diethylstiboestrol) which increases TRPV1 through mRNA upregulation, oleoylethanolamide, which sensitizes TRPV1, and AEA (a partial agonist of TRPV1), capzaizepin, norepinephrine, beta adrenergic agonists, diethylstiboestrol, oleoylethanolamide, and anandamide (AEA).

Another embodiment of the present disclosure provides methods of treating cannabinoid hyperemesis syndrome (CHS) with an agent that activates the endocannabinoid system (ECS) in the patient.

The endocannabinoid system (ECS) is composed of endocannabinoids, which are endogenous lipid-based retrograde neurotransmitters that bind to cannabinoid receptors, and cannabinoid receptor proteins that are expressed throughout the vertebrate central nervous system and peripheral nervous system. The endocannabinoid system is involved in regulating a variety of physiological and cognitive processes including pain-sensation, and in mediating the pharmacological effects of cannabis. It is discovered herein that activation of the ECS, or more specifically the activation of endocannabinoids such as anandamide (AEA), may be useful in reducing the symptoms of CHS.

It's observed that cigarette smoking was able to reduce nausea and vomiting in individuals suffering from CHS. In some embodiments, a method is provided for treating cannabinoid hyperemesis syndrome (CHS), which method entails administering to the person in need an effective amount of nicotine. The nicotine may be provided as a patch, a gum, a cream, or an injectable solution, without limitation.

In some embodiments, a method is provided for treating cannabinoid hyperemesis syndrome (CHS), which method entails administering to the person in need an effective amount of capsaicin or a mimetic, such as resiniferatoxin (RTX). RTX is functional analog of capsaicin that is more potent than capsaicin. The capsaicin or RTX may be administered as a cream, a capsule, an intranasal formulation, or an injectable solution, without limitation.

Agents that activate the ECS can also be administered endocannabinoids, such as anandamide (AEA), 2-arachidonoylglycerol (2-AG), and a mimetic or activator thereof. R (+)-methanandamide (mAEA), for instance, is a synthetic, non-hydrolysable anandamide analogue, and dihydroxyphenylglycine (DHPG) both can activate the ECS (Edwards et al. Hippocampus vol. 22, 2 (2010): 209-21).

Agents that activate the ECS can also be an AEA precursor, or one that activates AEA through TRPV1. In some embodiments, the agent is N-palmitoylethanolamide (PEA) or N-oleoylethanolamide (OEA) (Ho et al. British journal of pharmacology vol. 155, 6 (2008): 837-46)

Food supplements that activate the ECS can also be used, alone or in combination with any agent disclosed herein. Non-limiting examples include omega 6 fatty acids, estradiol, galantamine, DHA, testosterone, DHT, butyrate, extra virgin olive oil, resistant starch, calcium, potassium, folate, vitamin A, progesterone, cortisol, endorphins, epinephrine, oleamide, chocolate, N-acylethanolamines (NAEs), N-linoleoylethanolamide, N-oleoylethanolamine, palmitoylethanolamide, tea/EGCG, inositol, resveratrol, bile acid, hydrogen peroxide, honokoil, agmatine, genistein, kava/yangonin, kaempferol, 7-hydroxyflavone, 3,7-dihydroxyflavone, caffeine, flavonoids, caryophyllene, echinacea, black truffle, and diindolylmethane (DIM), Ruta Graveolens, Acmella Oleracea, Helichrysum Umbraculigerum, and Radula Marginata.

### Methods of Administration

In any of the methods described herein the TRPV1 antagonist and marijuana-derived cannabinoid composition may be administered together as a combination formulation or concurrently. With concurrent administration, the TRPV1 modulator can be administered via a different route than the marijuana-derived cannabinoid. For example, in certain embodiments, the TRPV1 modulator is administered orally and the marijuana-derived cannabinoid is administered via inhalation. In other embodiments, the TRPV1 modulator and the marijuana-derived cannabinoid are administered orally.

Each form of intake can be formulated to take advantage of those pathways as effectively as possible in delivering a therapeutic dose of the marijuana-derived cannabinoid as the medicinal effects are generally dependent on the route of administration and the quality of materials by which the form of intake was made and the quality and effectiveness of the “form.” In the methods described herein, the marijuana-derived cannabinoid can be administered via any suitable form of intake, and one or more times per day (e.g., once daily, twice daily, or three times daily).

The route of administration can affect the bioavailability, accuracy of dose (i.e., the degree to which one can quantify the amount of medicine in each dose), the time to onset (i.e., the amount of time before the medicinal or pharmacological effects are felt), the duration of effect (i.e., the length of time the medicinal or pharmacological express themselves), and the quality and consistency of the effect (i.e., the degree to which one can depend on a repeatable experience).

As used herein, the term “bioavailability” is intended to refer to the fraction of the administered dose that reaches systemic circulation; the higher the number, the larger fraction of the dose that reaches systemic circulation. (Note: 100% bioavailability is intravenous delivery and the standard by which bioavailability is defined and compared).

Routes (or forms) of administration suitable for use in the methods described herein include, but are not limited to, lungs (e.g., inhalation via smoking or vaporizing), oral (e.g., edibles or capsules), or transdermal (e.g., patches or sublingual drops).

Smoking is the inhalation of combusted, dried flowers of the cannabis plant. It is one of the quickest ways to feel the effects of medical marijuana and the most common form of intake. Typically, the onset is rapid, immediate, and the duration is short. this “peak effect” is due to the high bioavailability of cannabinoids in the smoke and rapid metabolizing of the cannabinoids in vivo.

Pipes, joints, and water pipes are all used to smoke medical cannabis; however, habitually smoking medical marijuana can lead to lung damage as well as a habituation of “high” caused by the rapid onset and diminishing dose-response, i.e. “peak effect,” which is why registered marijuana dispensaries will, when asked, either recommend vaporization or alternative methods of intake instead of smoking.

Vaporizing is a process whereby the dried cannabis flower or extract or concentrate is slowly subjected to heat at lower and more precise temperature allowing for the therapeutic ingredients in cannabis, cannabinoids such as THC, to phase-change into a gas or vapor and extract from the plant without burning the plant. This process produces vapor instead of smoke.

While reducing the harm of long-term cannabis use, vaporizing does not really change the absorption of cannabinoids into the body. The cannabinoids are taken in through the alveoli in the lungs like smoking. It is safer than inhaling smoke and allows the patient to better control the dose.

Infusing cannabis into butter or cooking oil or any other edible fat or sugar alcohol produces edible cannabis in the form of food or capsules. Edible consumption is occasionally recommended for patients who experience severe chronic pain because it's effects manifest in more of a “body high” than a “head high” and have been described as a deeper or heavier feeling.

Patients take caution with edible cannabis because it can sometimes take up to twenty minutes to an hour to take effect and can last for four hours or more. This is caused by a chemical change and loss of the molecular structure of THC during digestion and metabolism, a process known as first-pass metabolism.

A significant portion of the administered dose is destroyed due to the high PH of the stomach acids, and a part of the surviving portion of its molecules change into Δ¹¹-HydroxyTHC, which is a much more psychoactive metabolite of Δ⁹-THC that takes much longer to cross the blood-brain barrier. When eating an edible, this causes one to easily over consume and for one to have a more significant psychoactive effect. So, it is recommended that patients eat small portion of edible medical cannabis at a time—a form of dose monitoring known as “titration”—and wait an hour or so for it to take affect before deciding how to proceed.

Transdermal delivery is a self-contained, discrete dosage form which, when applied to intact skin, deliver the drug(s) through the skin at a controlled rate into systemic circulation. It comes in the form of a patch or a gel that is applied directly to the skin. The advantages of transdermal are many. The dose is pre-loaded and is hidden from view-no smell, no ritual, easy to use. The rate at which the dose is administered is consistent and reliable happening gradually over a set time course.

Sublingual products are the preferred method of treatment for many cancer patients who are taking full extract cannabis oil (sometimes referred to as “hemp oil” or Rick Simpson Oil). Direct sublingual application involves placing precisely decarboxylated cannabis under the tongue, allowing the cannabinoids to immediately enter the bloodstream through the vessel-rich tissues within the sublingual cavity. Additionally, sublingual delivery provides rapid effects similar to smoking without exposing the lungs to heat, tar, or other unwanted collateral effects, including unpleasant smoke smell, smoky taste, dry mouth, throat irritation and caused both by smoke and hot embers that often enter the user's mouth and lungs during administration.

In some embodiments, the compositions and/or methods are used to treat any one or more of the following: nausea and vomiting, wasting syndrome (AIDS), lack of appetite (exhibited in cancer and AIDs patients as well as patients suffering from anorexia nervosa), multiple sclerosis, spinal cord trauma, epilepsy, pain, arthritis (and other musculoskeletal disorders), movement disorders, glaucoma, asthma, hypertension, psychiatric disorders, Alzheimer's and dementia, general inflammation, gastrointestinal disorders.

In some embodiments, the CHS is treated by alleviating one or more symptoms of CHS. In certain embodiments, the symptoms include one or more of nausea (e.g., recurrent nausea), pain in the abdominal region (often described as “colicky”), vomiting (e.g., cyclical vomiting), and/or a compulsive urge to keep having hot water baths. In certain embodiments, the symptoms include one or more of anxiety, agitation, sweating, tachycardia, postural hypotension, polydipsia, and/or mild fever.

In certain embodiments, the marijuana-derived cannabinoid can be administered via a method different from the TRPV1 modulator. In certain embodiments, the TRPV1 modulator is administered orally and the marijuana-derived cannabinoid is administered via inhalation.

The daily dosage of marijuana-derived cannabinoid may be varied over a wide range; e.g., from about 10 to about 10,000 mg per adult human per day. For oral administration, the compositions are preferably provided in the form of an oral formulation containing about 0.1, 0.25, 0.5, 1.00, 5.00, 10.0, 15.0, 25.0, 50.0, 100, 200, 300, 400, 500, 600, 700, 800, 900 1000, 2000, 3000, 4000, 5000, 6000, 7000, 8000, 9000 or 10,000 milligrams of the active ingredient for the symptomatic adjustment of the dosage to the patient to be treated. The instant formulations typically contain from 10 mg to about 2000 mg, preferably, from about 50 mg to about 1000 mg of active ingredient. An effective amount of the instant compounds is ordinarily supplied at a dosage level of from about 0.002 mg/kg to about 150 mg/kg of body weight per day. Preferably, the range is from about 0.02 to about 80 mg/kg of body weight per day, and especially from about 0.2 mg/kg to about 40 mg/kg of body weight per day. The compositions may be administered on a regimen of about 1 to about 10 times per day.

The daily dosage of non-cannabinoid TRPV1 modulator may be varied over a wide range; e.g., from about 1 to about 1,000 mg per adult human per day. In certain embodiments, the method comprises administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be decreased daily, every other day, twice per week, or once per week.

It is understood that the dosage may vary widely from person to person based on numerous factors and can be determined by a clinician. In certain embodiments, toxicity and therapeutic efficacy of such therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. In certain embodiments, compounds exhibiting high therapeutic indices are preferred. In some embodiments, the data obtained from cell culture assays and animal studies is used in formulating a range of dosage for use in human. In specific embodiments, the dosage of such compounds lies within a range of circulating concentrations that include the ED₅₀ with minimal toxicity. In certain embodiments, the dosage varies within this range depending upon the dosage form employed and the route of administration utilized.

The pharmaceutical compositions described herein can be administered to a patient per se, or in pharmaceutical formulations where they are mixed with other active ingredients, as in combination therapy, or suitable pharmaceutically acceptable carriers or excipient(s). Techniques for formulation and administration of the compounds of the instant application may be found in “Remington's Pharmaceutical Sciences,” Mack Publishing Co., Easton, PA, 18th edition, 1990.

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A method of treating cannabinoid hyperemesis syndrome (CHS) in a patient, comprising administering to a patient in need thereof, at least one marijuana-derived cannabinoid in combination with a therapeutically effective amount of at least one TRPV1 antagonist.

2. The method of claim 1, wherein the TRPV1 antagonist is a competitive antagonist.

3. The method of claim 2, wherein the TRPV1 antagonist is selected from the group consisting of capsazepine, JYL-1421, A-425619, BCTC, JNJ-17203212, SB-705498, SB-366791, AMG-9810, MK-2295, and AMG-2674.

4. The method of claim 1, wherein the TRPV1 antagonist is a non-competitive antagonist.

5. The method of claim 4, wherein the TRPV1 antagonist is selected from the group consisting of an arginine-rich hexapeptide, methoctramine, AG-489, AG-505, DD-161515, and DD-191515.

6. The method of claim 1, wherein the TRPV1 antagonist and marijuana-derived cannabinoid are orally administered in a combination formulation.

7. The method of claim 1, further comprising at least partially activating TRPV1.

8. The method of claim 7, wherein the activating TRPV1 comprises nociceptive heat.

9. A method of treating cannabinoid hyperemesis syndrome (CHS) in a patient, comprising administering to a patient in need thereof a therapeutically effective amount of a TRPV1 upregulator.

10. The method of claim 9, wherein the TRPV1 upregulator is selected from the group consisting of capsazepine, norepinephrine, beta adrenergic agonists, diethylstiboestrol, oleoylethanolamide, and anandamide (AEA).

11. The method of claim 9, further comprising administering to the patient at least one marijuana-derived cannabinoid.

12. The method of claim 11, wherein the marijuana-derived cannabinoid composition comprises cannabidiol (CBD) and less than about 3% of a combination of cannabigerol, cannabichromene and cannabidiol acid.

13. A method of treating cannabinoid hyperemesis syndrome (CHS) in a patient, comprising administering to a patient in need thereof a therapeutically effective amount of an agent that activates the endocannabinoid system (ECS) in the patient.

14. The method of claim 13, wherein the agent comprises nicotine.

15. The method of claim 14, wherein the nicotine is administered as a patch, a gum, a cream, or an injectable solution.

16. The method of claim 13, wherein the agent comprises capsaicin or resiniferatoxin (RTX).

17. The method of claim 16, wherein the capsaicin or RTX is administered as a cream, a capsule, an intranasal formulation, or an injectable solution.

18. The method of claim 13, wherein the agent comprises an endocannabinoid.

19. The method of claim 13, wherein the agent is selected from the group consisting of anandamide (AEA), 2-arachidonoylglycerol (2-AG), and a mimetic or activator thereof.

20. The method of claim 19, wherein the agent is selected from the group consisting of R (+)-Methanandamide, dihydroxyphenylglycine (DHPG), N-palmitoylethanolamide (PEA), and N-oleoylethanolamide (OEA).
